(19) United States
(12) Patent Application Publication
(10) Pub. No.: US 2023/0360831 A1
(43) Pub. Date: Nov. 9, 2023

(54) SUPERCONDUCTING COIL APPARATUS, SUPERCONDUCTING ACCELERATOR, AND PARTICLE BEAM THERAPY APPARATUS

(71) Applicants: TOSHIBA ENERGY SYSTEMS & SOLUTIONS CORPORATION, Kawasaki-shi (JP); National Institutes for Quantum Science and Technology, Chiba-shi (JP)

(72) Inventors: Shigeki TAKAYAMA, Yokohama Kanagawa (JP); Tomofumi ORIKASA, Yokohama Kanagawa (JP); Kota MIZUSHIMA, Narashino Chiba (JP); Yoshiyuki IWATA, Narashino Chiba (JP); Yasushi ABE, Koshigaya Saitama (JP); Tetsuya FUJIMOTO, Yachiyo Chiba (JP)

(73) Assignees: TOSHIBA ENERGY SYSTEMS & SOLUTIONS CORPORATION, Kawasaki-shi (JP); National Institutes for Quantum Science and Technology, Chiba-shi (JP)

(21) Appl. No.: 18/352,712

(22) Filed: Jul. 14, 2023

Related U.S. Application Data
(63) Continuation of application No. PCT/JP22/05186, filed on Feb. 9, 2022.

(30) Foreign Application Priority Data
Mar. 23, 2021 (JP) 2021-048606

(51) Int. Cl. H01F 6/06; H05H 7/00

(52) U.S. Cl. CPC H01F 6/06; H05H 7/001; A61N 2/02

(57) ABSTRACT

According to one embodiment, a superconducting coil apparatus comprising at least one superconducting coil formed of a plurality of turns under a definition that one turn is a portion of a superconducting wire annularly wound for one round, wherein: the superconducting coil has a shape along an outer peripheral surface of a tubular structure having a tubular shape; each of the plurality of turns has a coil longitudinal portion extending along an axial direction of the tubular structure and a coil end portion extending from the coil longitudinal portion along a circumferential direction of the tubular structure; and a boundary line indicating a border between the coil longitudinal portion and the coil end portion at each of the plurality of turns is inclined with respect to a reference line extending in the circumferential direction of the tubular structure in a side view of the tubular structure.

### CROSS-REFERENCE TO RELATED APPLICATIONS

This application is a Continuation Application of No. PCT/JP2022/005186, filed on Feb. 9, 2022, and the PCT application is based upon and claims the benefit of priority from Japanese Patent Application No. 2021-048606, filed on Mar. 23, 2021, the entire contents of which are incorporated herein by reference.

### TECHNICAL FIELD

Embodiments of the present invention relate to a superconducting technique.

### BACKGROUND

Attention has been focused on a particle beam therapy technique in which a diseased tissue (cancer) of a patient is irradiated with a particle beam such as a carbon ion beam for treatment. This particle beam therapy technique can pinpoint and kill only the diseased tissue without damaging normal tissues. Thus, the burden on the patient is less than surgery or medication treatment, and earlier return to society after the treatment can also be expected. In order to treat cancer cells deep inside the body, it is necessary to accelerate the particle beam. In general, particle beam accelerators configured to accelerate the particle beam are broadly classified into two types. One of the two types is a linear accelerator in which accelerating apparatuses are arranged in a straight line. The other type is a circular accelerator in which deflectors configured to bend the trajectory of the particle beam are arranged circularly and an accelerator is disposed in part of this circular trajectory. In particular, in the case of using heavy particles such as carbons and protons, it is common to adopt a method in which a linear accelerator is used for acceleration in the low energy band immediately after generation of the beam and a circular accelerator is used for acceleration in the high energy band.

The circular accelerator configured to circulate and accelerate the particle beam is configured by sequentially arranging: a quadrupole electromagnet configured to control the shape of the particle beam; a bending electromagnet configured to bend the trajectory of the particle beam; a steering electromagnet configured to correct deviation of the beam trajectory; and the like. In such an accelerator, when mass or energy of the particles to be circulated increases, magnetic rigidity (i.e., difficulty of bending by the magnetic field) increases, and thus the beam trajectory radius increases. As a result, the entirety of the apparatus becomes larger in size. As the size of the apparatus increases, the incidental facilities such as the building also increase in size, and consequently, this apparatus cannot be introduced in a place where the installation range is limited, such as in an urban site. Further, in order to suppress increase in size of the apparatus, it is necessary to increase the magnetic field strength to be generated by the bending electromagnet. In a general bending electromagnet, influence of magnetic saturation of its iron core makes it difficult to generate a magnetic field exceeding 1.5 T. For this reason, it is desired to apply a superconducting technique, which can achieve both high magnetic field and miniaturization of the circular accelerator, to a bending electromagnet.

### PRIOR ART DOCUMENT

### Patent Document

[Patent Document 1] JP H10-144521 A
[NON-Patent Document 1] “Field Computation for Accelerator Magnets” Stephan Russenschuck WiLEY-VCH

### SUMMARY

### Problems to be Solved by Invention

In general, a saddle type coil is used as a conventional superconducting coil for an accelerator. In the conventional technology, spacers (gaps) are provided between the superconducting wires at the ends of the coils in order to generate a uniform magnetic field, i.e., in order to reduce high-order multipolar components. Hence, there is a problem that the coil ends are extended and the superconducting coil becomes larger in size.

Further, in the conventional technology, there is also a method of adding a correction coil in order to cancel an undesired magnetic field to be generated at the coil ends. Also in this method, it is necessary to overlap the correction coil outside the main coil, and thus, there is a problem that the size of the superconducting coil becomes larger in a radial direction, in an axial direction, or in both directions.

In view of the above-described circumstances, an object of embodiments of the present invention is to provide a superconducting technique by which size of a superconducting coil apparatus can be reduced.

### BRIEF DESCRIPTION OF DRAWINGS

FIG. 1 is a conceptual diagram illustrating a particle beam therapy apparatus according to the present embodiment.

FIG. 2 is a plan view of a circular accelerator.

FIG. 3 is a plan view of a superconducting coil of a first layer.

FIG. 4 is a side view of the superconducting coil of the first layer.

FIG. 5 is a cross-sectional view taken along the line V-V of FIG. 4.

FIG. 6 is a plan view of a superconducting coil of a second layer.

FIG. 7 is a side view of the superconducting coil of the second layer.

FIG. 8 is a cross-sectional view taken along the line VIII-VIII of FIG. 7.

FIG. 9 is a side view illustrating a state in which the turns of the first layer and the second layer are overlapped.

FIG. 10 is an exploded perspective view of a superconducting coil according to a modification.

FIG. 11 is a plan view of a conventional superconducting coil.

### DETAILED DESCRIPTION

In one embodiment of the present invention, a superconducting coil apparatus comprising at least one superconducting coil formed of a plurality of turns under a definition that one turn is a portion of a superconducting wire annularly wound for one round, wherein: the superconducting coil has a shape along an outer peripheral surface of a tubular structure having a tubular shape; each of the plurality of turns has a coil longitudinal portion extending along an axial direction of the tubular structure and a coil end portion extending from the coil longitudinal portion along a circumferential direction of the tubular structure; and a boundary line indicating a border between the coil longitudinal portion and the coil end portion at each of the plurality of turns is inclined with respect to a reference line extending in the circumferential direction of the tubular structure in a side view of the tubular structure.

According to embodiments of the present invention, it is possible to provide a superconducting technique by which size of a superconducting coil apparatus can be reduced.

Hereinbelow, a description will be given of embodiments of a superconducting coil apparatus, a superconducting accelerator, and a particle beam therapy apparatus in detail by referring to the accompanying drawings.

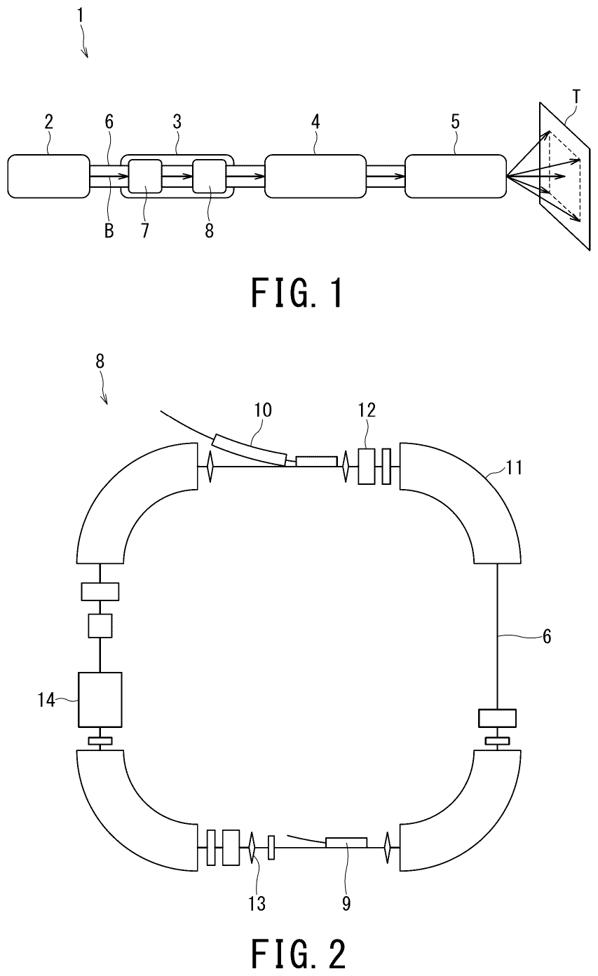

The reference sign 1 in FIG. 1 denotes a particle beam therapy apparatus according to the present embodiment. The particle beam therapy apparatus 1 is a beam irradiation apparatus that accelerates a particle beam B and irradiates a lesion site T, i.e., the target with this particle beam B for treatment.

The particle beam therapy apparatus 1 uses charged particles (for example, negative pions, protons, helium ions, carbon ions, neon ions, silicon ions, and argon ions) as the particle beam B for therapeutic irradiation.

The particle beam therapy apparatus 1 includes: a beam generator 2; a beam accelerator 3; a beam transport apparatus 4; a beam irradiator 5; and vacuum duct 6, by which these components are connected and through which the particle beam B passes.

The vacuum duct 6 maintains their inside in a vacuum state. The particle beam B passes through the inside of the vacuum duct 6, and thereby, beam-loss due to scattering between the particle beam B and air is suppressed. The vacuum duct 6 extends to just before the position of the lesion site T of the patient. The particle beam B having passed through the vacuum duct 6 is radiated onto the lesion site T of the patient.

The beam generator 2 is an apparatus that generates the particle beam B and is, for example, an apparatus that extracts ions generated by using an electromagnetic field, a laser, or the like.

The beam accelerator 3 is disposed on the downstream side of the beam generator 2. The beam accelerator 3 is an apparatus that accelerates the particle beam B to a predetermined energy. The beam accelerator 3 is composed of a front-stage accelerator and a rear-stage accelerator, for example. As the front-stage accelerator, a linear accelerator 7 configured of a drift tube linac (DTL) or a radio-frequency quadrupole linear accelerator (RFQ) is used. As the rear-stage accelerator, a circular accelerator 8 configured of a synchrotron or a cyclotron is used. The beam trajectory of the particle beam B is formed by the linear accelerator 7 and the circular accelerator 8.

The beam transport apparatus 4 is disposed on the downstream side of the beam accelerator 3. The beam transport apparatus 4 is an apparatus configured to transport the accelerated particle beam B to the patient's lesion site T as the irradiation target. The beam transport apparatus 4 is composed of deflectors, focusing/defocusing apparatuses, a hexapole apparatus, a beam trajectory correction apparatus, its controller, and the like which are arranged around the vacuum duct 6.

The beam irradiator 5 is disposed at the downstream of the beam transport apparatus 4. The beam irradiator 5 controls the beam trajectory of the particle beam B and monitors the irradiation position and irradiation dose of the particle beam B at the lesion site T so as to accurately irradiate the irradiation point having been set at the lesion site T of the patient with the particle beam B, which has passed through the beam transport apparatus 4 and has a predetermined energy.

In the beam accelerator 3 and the beam transport apparatus 4, a superconducting technique which can achieve both the high magnetic field and miniaturization is used. In the present embodiment, the circular accelerator 8 of the beam accelerator 3 is illustrated as an application aspect of the superconducting technique. In other words, the particle beam therapy apparatus 1 according to the present embodiment includes the circular accelerator 8 as a superconducting accelerator. At least part of the beam trajectory for accelerating the particle beam B is formed by the circular accelerator 8.

As shown in FIG. 2, the circular accelerator 8 as the superconducting accelerator of the present embodiment is built along the vacuum duct 6, which are formed annularly (i.e., approximately in a circular shape) in plan view. The circular accelerator 8 includes: a beam injector 9; a beam emitter 10; deflectors 11; focusing/defocusing apparatuses 12; hexapole apparatuses 13; and an acceleration-force application apparatus 14.

The circular accelerator 8 circulates the particle beam B along the vacuum duct 6 by deflecting the trajectory of the particle beam B injected from the linear accelerator 7 via the beam injector 9 with the use of the deflectors 11. The particle beam B is stably circulated by using the focusing/defocusing apparatuses 12 and the hexapole apparatuses 13.

When the particle beam B circulates the beam trajectory of the circular accelerator 8, acceleration force is applied to the particle beam B by the acceleration-force application apparatus 14. Further, the particle beam B is accelerated to a predetermined energy, and this accelerated particle beam B is emitted from the beam emitter 10 so as to reach the lesion site T.

Although the deflectors 11 deflect the particle beam B by the magnetic field in the circular accelerator 8, when the mass or energy of the particles to be circulated is increased, the magnetic rigidity (i.e., difficulty in bending by the magnetic field) is increased, and thereby the beam trajectory radius becomes larger. As a result, the circular accelerator 8 becomes larger in size as a whole. In order to suppress the increase in size of the circular accelerator 8, it is necessary to increase the magnetic field strength to be generated by the deflectors 11. In the present embodiment, magnetic field strength can be enhanced and the size of the circular accelerator 8 can be reduced by applying a superconducting technology to the deflectors 11.

Here, superconducting wires are composed of: low-temperature superconductors such as NbTi, Nb₃Sn, Nb₃Al, and MgB₂; and high-temperature superconductors such as a Bi₂Sr₂Ca₂Cu₃O₁₀ wire and a REB₂C₃O₇ wire.

In the above notation, “RE” in “REB₂C₃O₇” means at least one of rare earth elements (for example, neodymium (Nd), gadolinium (Gd), holmium (Ho), and samarium (Sm)) and yttrium elements. Additionally, “B” means barium (Ba), “C” means copper (Cu), and “0” means oxygen (O).

In the case of using the low-temperature superconductors, a curved surface can be readily formed because the low-temperature superconductors have ductility. In the case of using the high-temperature superconductors, the superconducting state occurs at high temperatures, which reduces the cooling load and improves the operating efficiency.

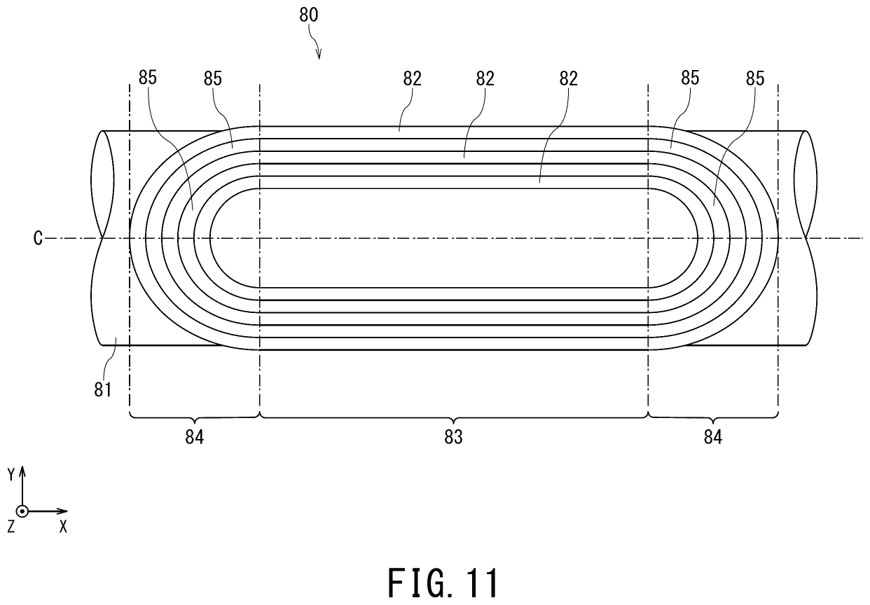

Next, a conventional general superconducting coil 80 will be described by referring to FIG. 11. The superconducting coil 80 is provided on a side surface of a cylindrical tubular structure 81. This superconducting coil 80 includes a plurality of conductor portions 82 around which superconducting wires are wound. Each conductor portion 82 is divided into a coil longitudinal portion 83 and coil end portions 84. In the coil longitudinal portion 83, the spacings in the circumferential direction between the conductor portions 82 are not uniform, and desired magnetic field distribution is generated in the beam passage region at the central portion of the superconducting coil 80 depending on the distance.

Here, current density distribution corresponding to a magnetic field to be generated by the general superconducting coil 80 will be described. In a cross-sectional view of the tubular structure 81, a predetermined position in the circumferential direction of the tubular structure 81 is represented by an angle θ of the central axis.

For example, in the case of generating a dipole magnetic field that is a uniform magnetic field, the conductor portions 82 in the coil longitudinal portion 83 are arranged in such a manner that the current density distribution is close to a function of cos θ. Similarly, in the case of generating a quadrupole magnetic field, the conductor portions 82 in the coil longitudinal portion 83 are arranged in such a manner that the current density distribution is close to a function of cos 2θ. In the case of generating a hexapole magnetic field, the conductor portions 82 in the coil longitudinal portion 83 are arranged in such a manner that the current density distribution is close to a function of cos 3θ. In the case of generating an octupole magnetic field, the conductor portions 82 in the coil longitudinal portion 83 are arranged in such a manner that the current density distribution is close to a function of cos 4θ.

Each coil end portion 84 has a three-dimensional shape along the surface of the tubular structure 81 so that the conductor portions 82 forming the coil end portions 84 do not physically block the beam passage region. Thus, each coil end portion 84 has a shape in which the conductor gradually transitions from the side surface to the top surface of the tubular structure 81.

In each coil end portion 84, current density distribution different from the current density distribution to be generated in the coil longitudinal portion 83 is generated. Thus, an error magnetic field (unnecessary magnetic field component) being disturbed from the desired magnetic field distribution is generated. For example, in the case of generating a dipole magnetic field, at each coil end portion 84, the conductor portion 82 changes from the position of θ=0° to the position of θ=90°. At this time, the current density distribution such as cos 2θ or cos 3θ is superimposed on the current density distribution of cos θ. Thus, a negative hexapole magnetic field (hexapole component) is generated.

In the conventional technology, spacers 85 (gaps) are provided for the coil end portions 84 in order to suppress this negative hexapole magnetic field. Further, a positive hexapole magnetic field is generated by maintaining the conductor portions 82 provided near the position of θ=0°, and thereby a desired uniform magnetic field is obtained. However, in this method, the coil end portions 84 are extended, thereby the overall dimension of the superconducting coil 80 is increased, and consequently, the overall size of the circular accelerator 8 is increased. For this reason, in the present embodiment, a desired uniform magnetic field is obtained and the size of the superconducting coil 80 is reduced by appropriately arranging the superconducting wires.

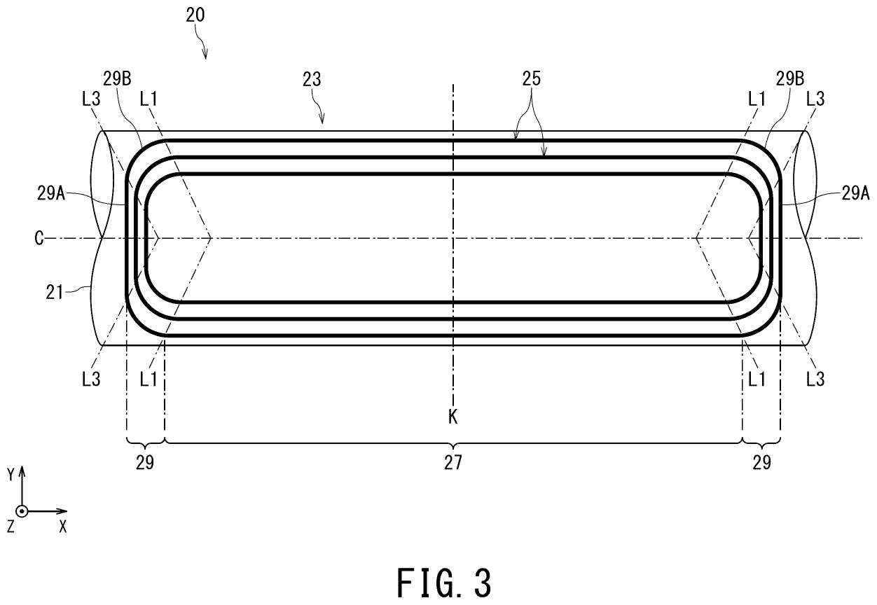

Next, a description will be given of the superconducting coil apparatus 20 provided in the circular accelerator 8 as the superconducting accelerator of the present embodiment by using FIG. 3 to FIG. 9. In the following description regarding the superconducting coil apparatus 20, when an axial direction in which the particle beam B passes (i.e., the direction in which the axis C extends) is defined an X-axis direction, the state of the superconducting coil apparatus 20 as viewed from a Y-axis direction is defined as a side view, and the state of the superconducting coil apparatus 20 as viewed from a Z-axis direction is defined as a plan view (top view). Although the superconducting coil apparatus 20 is not an apparatus to be affected by gravity and there is no distinction between which side is up and down, it is assumed for convenience that the Z-axis direction is the upward direction of the superconducting coil apparatus 20.

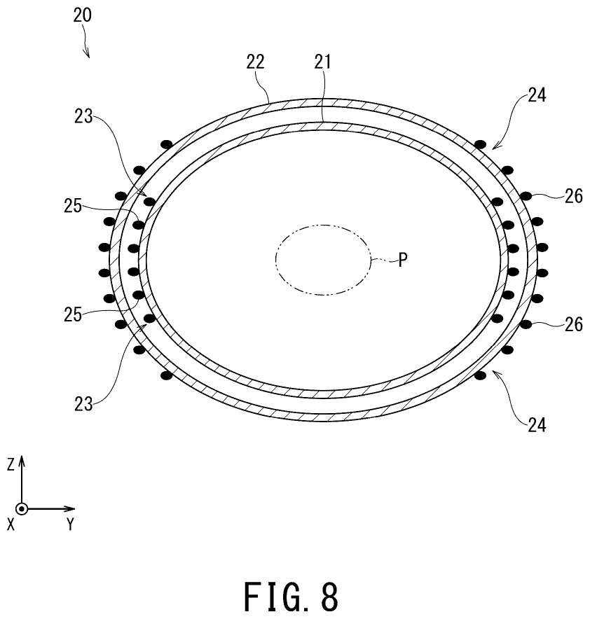

First, as shown in FIG. 8, the superconducting coil apparatus 20 of the present embodiment has a two-layer structure. This superconducting coil apparatus 20 includes: a first-layer tubular structure 21 that is disposed on the innermost circumference and forms a tubular shape; and a second-layer tubular structure 22 that is disposed on the outer circumference of the first-layer tubular structure 21 and forms a tubular shape. These tubular structures 21 and 22 are arranged concentrically around the axis C, in other words, arranged coaxially with each other.

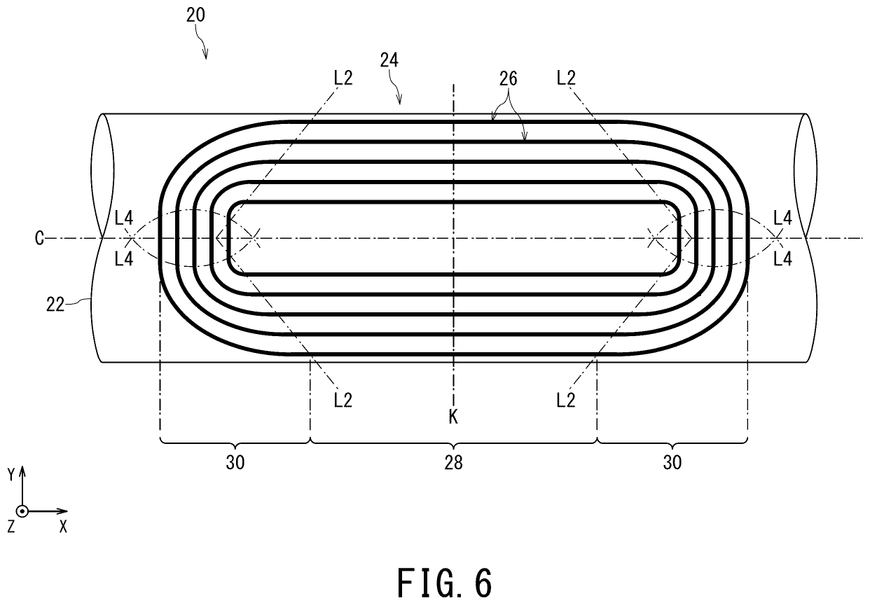

As shown in FIG. 3 to FIG. 5, the superconducting coil apparatus 20 includes two superconducting coils 23 provided above and below the tubular structure 21 of the first layer. As shown in FIG. 6 to FIG. 8, the superconducting coil apparatus 20 includes two superconducting coils 24 provided above and below the tubular structure 22 of the second layer. In other words, at least two superconducting coils 23 and 24 are laminated in a radial direction of the tubular structures 21 and 22. These superconducting coils 23 and 24 can generate a magnetic field in the passage region P of the particle beam B.

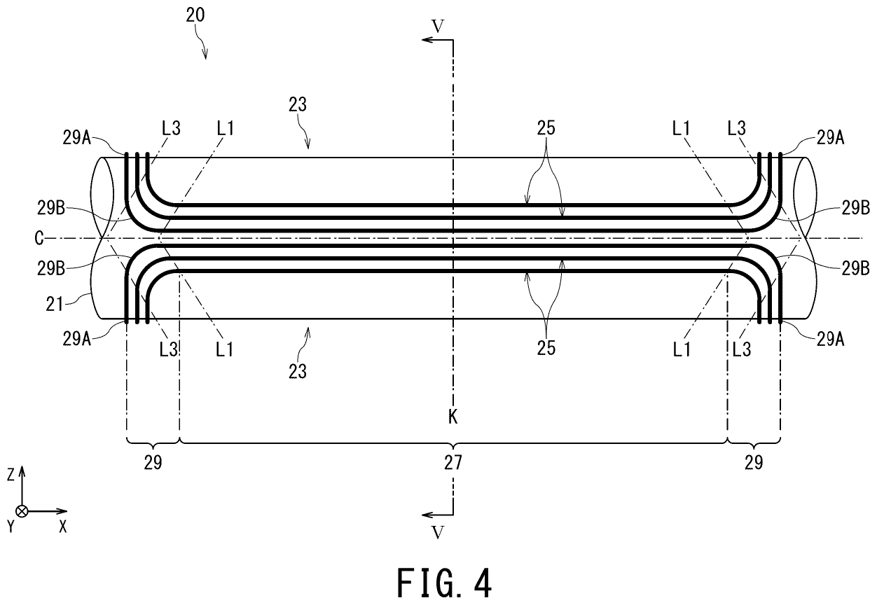

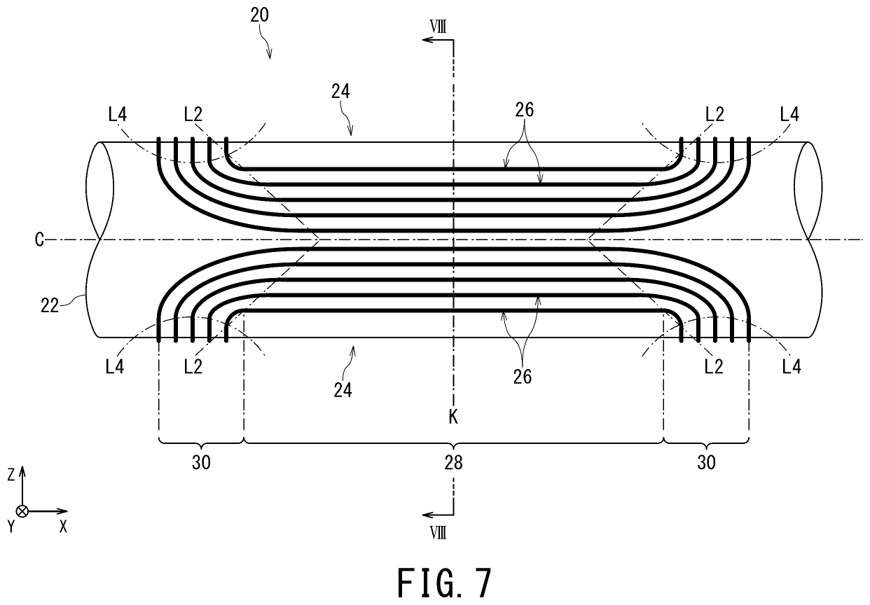

As shown in FIG. 8, layers of the two superconducting coils 23 and 24 are provided in the upper halves of the tubular structures 21 and 22 of the respective layers, and layers of the superconducting coils 23 and 24 are provided in the lower halves of the tubular structures 21 and 22 of the respective layers (FIG. 4 and FIG. 7).

Each of the superconducting coils 23 and 24 has a shape along the outer peripheral surface of the tubular structure 21 or 22. The tubular structures 21 and 22 are members that support the superconducting coils 23 and 24. The tubular structure 21 of the innermost first layer is disposed at the axis C of the superconducting coil apparatus 20. This first-layer tubular structure 21 forms part of the vacuum duct 6. This tubular structure 21 may be a separate member from the vacuum duct 6. In other words, the vacuum duct 6 may be provided inside the tubular structure 21.

The superconducting coils 23 and 24 are formed by winding superconducting wires into an annular shape. For example, when one turn 25 (or 26) is defined as the portion of the superconducting wire wound for one round, one superconducting coil 23 (or 24) is formed by a plurality of turns 25 (or 26). In order to facilitate understanding, FIG. 3 shows a case where three turns 25 form one superconducting coil 23. FIG. 6 shows a case where five turns 26 form one superconducting coil 24. Actually, one superconducting coil 23 or 24 is formed by tens to hundreds of turns 25 or 26.

Since the tubular structure 22 of the second layer has a wider outer peripheral surface than the tubular structure 21 of the first layer, more turns 26 can be arranged in the superconducting coil 24 of the second layer than in the superconducting coil 23 of the first layer.

The superconducting coil apparatus 20 is applied to, for example, the deflectors 11 of the circular accelerator 8 (FIG. 2). The deflectors 11 are provided with the vacuum duct 6 curved with a constant curvature. Thus, the tubular structures 21 and 22 to be used in the actual superconducting coil apparatus 20 are also members curved with a constant curvature. However, in order to facilitate understanding, in FIG. 3, FIG. 4, FIG. 6, and FIG. 7, the tubular structures 21 and 22 are shown as straight members. Similarly, the axis C of each of the tubular structures 21 and 22 is actually curved with a constant curvature but is shown as a straight line.

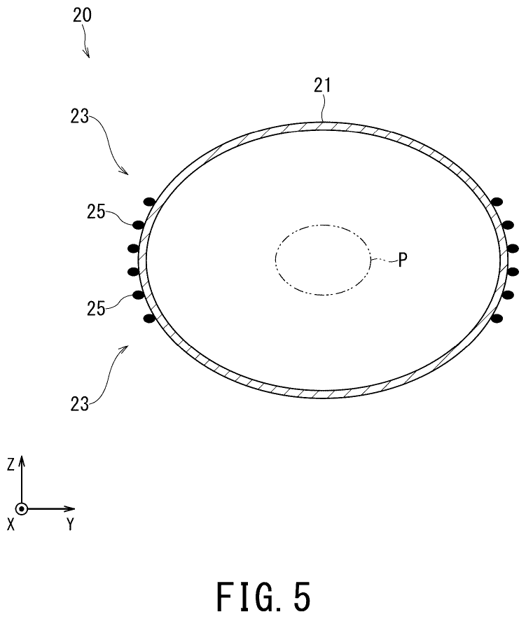

As shown in FIG. 5 and FIG. 8, the tubular structures 21 and 22 are elliptic in cross-section. For example, when the tubular structures 21 and 22 are curved in the Y-axis direction, each of the tubular structures 21 and 22 has an elliptic shape in which the diameter in the Y-axis direction is larger than the diameter in the Z-axis direction. In other words, the tubular structures 21 and 22 are elliptic in shape with increasing diameter in the curving direction. In this manner, the superconducting coil apparatus 20 can generate a magnetic field suitable for the bending direction of the particle beam B.

As shown in FIG. 4 and FIG. 7, in the superconducting coils 23 and 24, each turn 25 (or 26) includes: a coil longitudinal portion 27 (or 28) extending linearly along the axial direction (i.e., the X-axis direction) of the tubular structure 21 (or 22); and coil end portions 29 (or 30) extending from the coil longitudinal portion 27 (or 28) along the circumferential direction of the tubular structures 21 (or 22).

In the present embodiment, in a side view of the tubular structure 21 (or 22), boundary lines L1 (or L2) for demarcating the border between the coil longitudinal portion 27 (or 28) and the coil end portions 29 (or 30) at the respective turns 25 (or 26) are inclined with respect to a reference line K extending in the circumferential direction of the tubular structure 21 (or 22).

As shown in FIG. 3, in each turn 25 of the superconducting coil 23 of the first layer, the coil longitudinal portion 27 becomes shorter from the outer periphery side to the inner periphery side of the superconducting coil 23. Thus, the boundary line L1 becomes inclined with respect to the reference line K.

As shown in FIG. 6, in each turn 26 of the superconducting coil 24 of the second layer, the coil longitudinal portion 28 becomes longer from the outer periphery side to the inner periphery side of the superconducting coil 24. Thus, the boundary line L2 becomes inclined with respect to the reference line K.

In this manner, the coil longitudinal portions 27 and 28 of the superconducting coils 23 and 24 of the respective layers can change the aspect of the magnetic field to be generated at their ends.

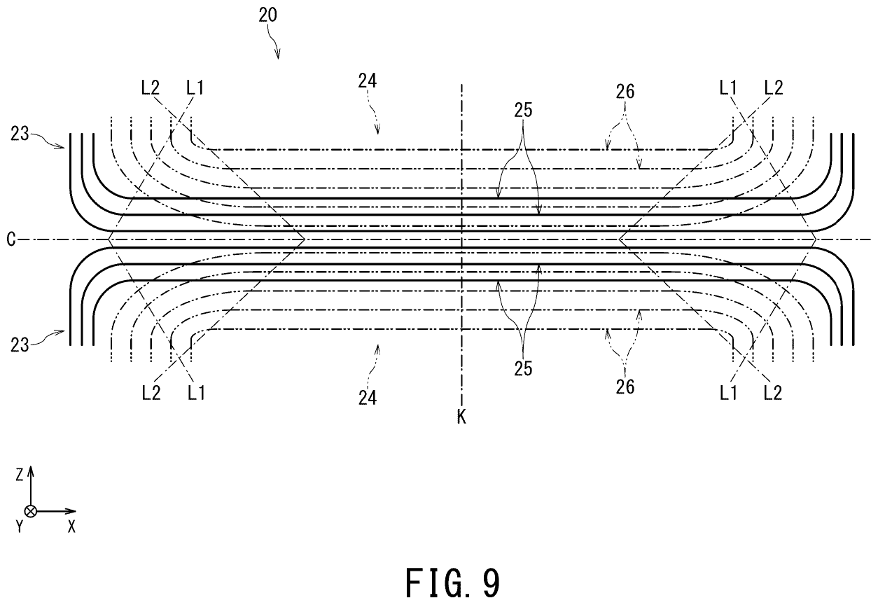

As shown in FIG. 9, in a side view of the tubular structure 21 and 22, the boundary line L1 of the superconducting coil 23 of the first layer and the boundary line L2 of the superconducting coil 24 of the second layer are provided so as to be displaced from each other. In the present embodiment, the positions of the boundary line L1 and the boundary line L2 are different in the axial direction (i.e., the X-axis direction). In this manner, an appropriate magnetic field can be formed by the superconducting coil 23 of the first layer and the superconducting coil 24 of the second layer.

In the present embodiment, the boundary line L1 of the superconducting coil 23 of the first layer and the boundary line L2 of the superconducting coil 24 of the second layer are inclined with respect to the reference line K in the directions opposite to each other. In this manner, the magnetic fields to be generated at the ends of the superconducting coil 23 of the first layer and the magnetic fields to be generated at the ends of the superconducting coil 24 of the second layer have different forms.

In the axial direction (i.e., the X-axis direction), the dimension of the superconducting coil 23 of the first layer is longer than the dimension of the superconducting coil 24 of the second layer. In other words, the ends of the superconducting coils 23 of the first layer protrude from the ends of the superconducting coils 24 of the second layer. Thus, each boundary line L1 of the superconducting coil 23 of the first layer is provided at a position closer to the end than each boundary line L2 of the superconducting coil 24 of the second layer.

The superconducting coil apparatus 20 of the present embodiment can suppress occurrence of an error magnetic field disturbed from the desired magnetic field distribution near the ends of the superconducting coils 23 and 24. For example, the error magnetic field at each end of the superconducting coil 23 of the first layer can be canceled by the magnetic field to be generated at each end of the superconducting coil 24 of the second layer.

As shown in FIG. 4 and FIG. 7, each coil end portion 29 (or 30) of the superconducting coil 23 (or 24) of each layer includes: a straight portion 29A (or 30A) extending linearly along the circumferential direction of the tubular structure 21 (or 22); and a bent portion 29B (or 30B) that is bent between the straight portion 29A (or 30A) and the coil longitudinal portion 27 (or 28). The straight portions 29A of the turns 25 adjacent to each other in the first layer are brought into close contact with each other. Further, the straight portions 30A of the turns 26 adjacent to each other in the second layer are brought into close contact with each other.

As shown in FIG. 4, each boundary line L3 indicating the border between the straight portion 29A and the bent portion 29B of each coil end portion 29 of the superconducting coil 23 of the first layer is a straight line. These boundary lines L3 are tilted with respect to the reference line K.

As shown in FIG. 7, each boundary line L4 indicating the border between the straight portion 30A and the bent portion 30B of each coil end portion 30 of the superconducting coil 24 of the second layer is curved. These boundary lines L4 are tilted with respect to the reference line K.

In the present embodiment, at the straight portions 29A and 30A of the coil end portions 29 and 30, the respective turns 25 and 26 (superconducting wires) can be densely arranged. Thus, the width (i.e., length in the X-axis direction) of the coil end portions 29 and 30 can be reduced.

As shown in FIG. 4, in order to shorten each coil end portion 29 of the first layer, the turns 25 (i.e., superconducting wires) are arranged densely, so the curvature (i.e., rising bend radius) of the bent portion 29B of each coil end portion 29 of the first layer is reduced. Thus, at each coil end portion 29, the current density distribution becomes a distribution in which a shape closes to the function of cos θ is superimposed. As a result, a positive hexapole magnetic field is generated. Here, as shown in FIG. 7, the curvature (i.e., rising bending radius) of the bent portion 30B of each coil end portion 30 of the second layer is increased. Thus, a negative hexapole magnetic field is generated and can cancel the positive hexapole magnetic field.

In this manner, the hexapole magnetic field can be suppressed by optimally setting the rising radii in the respective coil end portions 29 and 30 of the first and second layers. In addition, the curvatures of the bent portions 29B and 30B in the coil end portions 29 and 30 may be different between the turns 25 and 26 in the same layer. In this manner, the hexapole magnetic field can be suppressed. Further, the curvatures of the bent portions 29B and 30B in the coil end portions 29 and 30 may be different not only between the turns 25 and 26 in the same layer but also between respective layers. In this manner, the hexapole magnetic field can be suppressed in the entire superconducting coil apparatus 20.

Since the plurality of superconducting coils 23 and 24 are laminated in the radial direction of the tubular structures 21 and 22, many turns 25 and 26 (i.e., superconducting wires) can be arranged in the circumferential direction in a cross-sectional view of the tubular structures 21 and 22. Thus, a stronger magnetic field can be generated. As the tubular structures 21 and 22 are laminated in the radial direction, the outer circumference length is enlarged, so more turns 26 can be arranged in the outer layer (i.e., the second layer) than in the inner layer (i.e., the first layer). A stronger magnetic field can be generated by arranging many turns 25 and 26 with smaller number of layers.

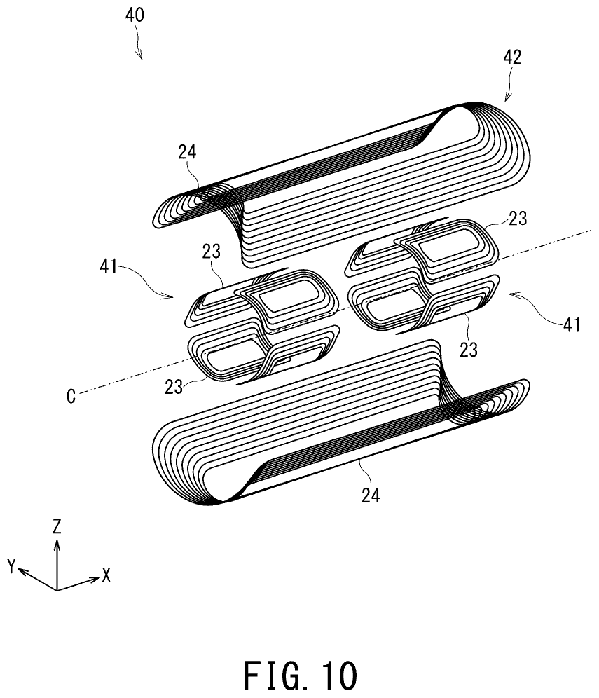

Next, a description will be given of a superconducting coil apparatus 40 of a modification by referring to FIG. 10. In an exploded perspective view of FIG. 10, in order to facilitate understanding, illustration of the tubular structure is omitted, and only the arrangement aspect of the superconducting coils 23 and 24 is illustrated.

The superconducting coil apparatus 40 of the modification includes: two superconducting quadrupole coils 41 provided in the first layer for generating a quadrupole magnetic field; and one superconducting dipole coil 42 provided in the second layer for generating a dipole magnetic field.

Each superconducting quadrupole coil 41 is formed by four superconducting coils 23. The two superconducting quadrupole coils 41 are arranged side by side in the axial direction (i.e., the X-axis direction).

The one superconducting dipole coil 42 is formed by two superconducting coils 24. The superconducting dipole coil 42 and the superconducting quadrupole coils 41 are arranged coaxially with each other.

The superconducting coil apparatus 40 of the modification can appropriately control the particle beam B with: a dipole magnetic field to be generated by the superconducting dipole coil 42; and a quadrupole magnetic field to be generated by the superconducting quadrupole coils 41.

Although the tubular structures 21 and 22 have an elliptic shape in cross-section in the above-described embodiments, other aspects may be adopted. For example, the tubular structures 21 and 22 may have a perfect circular shape or an oval shape as viewed in cross-section.

Although the tubular structures 21 and 22 have an elliptic shape in which the diameter increases in the bending direction in the above-described embodiments, other aspects may be adopted. For example, the tubular structures 21 and 22 may have an elliptic shape in which the diameter decreases in the bending direction.

Although the boundary lines L1, L2, and L3 are shown as straight lines in the above-described embodiments, other aspects may be adopted. For example, each boundary line L1, L2, or L3 may be curved or may be a mixture of a straight line and a curved line.

Although the aspect in which the boundary line L4 is curved is illustrated in the above-described embodiments, other aspects may be adopted. For example, the boundary line L4 may be a straight line or may be a mixture of a straight line and a curved line.

Although the boundary line L1 of the superconducting coil 23 of the first layer and the boundary line L2 of the superconducting coil 24 of the second layer are inclined in opposite directions with respect to the reference line K in the above-described embodiments, other aspects may be adopted. For example, the boundary line L1 of the superconducting coil 23 of the first layer and the boundary line L2 of the superconducting coil 24 of the second layer may be inclined in the same direction with respect to the reference line K.

According to the above-described embodiments, the boundary line indicating the border between the coil longitudinal portion and the coil end portion in each turn is inclined with respect to the reference line extending in the circumferential direction of the tubular structure, and this configuration can reduce the size of the superconducting coil apparatus.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

1. A superconducting coil apparatus comprising at least one superconducting coil formed of a plurality of turns under a definition that one turn is a portion of a superconducting wire annularly wound for one round, wherein:
the superconducting coil has a shape along an outer peripheral surface of a tubular structure having a tubular shape;
each of the plurality of turns has a coil longitudinal portion extending along an axial direction of the tubular structure and a coil end portion extending from the coil longitudinal portion along a circumferential direction of the tubular structure; and
a boundary line indicating a border between the coil longitudinal portion and the coil end portion at each of the plurality of turns is inclined with respect to a reference line extending in the circumferential direction of the tubular structure in a side view of the tubular structure.

2. The superconducting coil apparatus according to claim 1, wherein the coil longitudinal portion becomes shorter from an outer peripheral side to an inner peripheral side of the superconducting coil.

3. The superconducting coil apparatus according to claim 1, wherein the coil longitudinal portion becomes longer from an outer peripheral side to an inner peripheral side of the superconducting coil.

4. The superconducting coil apparatus according to claim 1, wherein:
the at least one superconducting coil comprises at least two the superconducting coils, the at least two superconducting coils are laminated in a radial direction of the tubular structure; and
the boundary line of the superconducting coil of a first layer and the boundary line of the superconducting coil of a second layer are displaced from each other in a side view of the tubular structure.

5. The superconducting coil apparatus according to claim 4, wherein, in a side view of the tubular structure, the boundary line of the superconducting coil of the first layer and the boundary line of the superconducting coil of the second layer are inclined with respect to the reference line in directions opposite to each other.

6. The superconducting coil apparatus according to claim 4, wherein:
the coil longitudinal portion becomes shorter from an outer peripheral side to an inner peripheral side of the superconducting coil of the first layer; and
the coil longitudinal portion becomes longer from an outer peripheral side to an inner peripheral side of the superconducting coil of the second layer.

7. The superconducting coil apparatus according to claim 1, wherein the coil end portion includes:
a straight portion extending linearly along the circumferential direction of the tubular structure; and
a bent portion that is bent between the straight portion and the coil longitudinal portion, and
wherein straight portions of adjacent turns are closely spaced.

8. The superconducting coil apparatus according to claim 1, wherein the tubular structure is curved with a constant curvature and has an elliptic shape in cross-section.

9. The superconducting coil apparatus according to claim 1, wherein the at least one superconducting coil comprises a plurality of the superconducting coils,
the superconducting coil apparatus further comprising:
a superconducting dipole coil that is formed of the plurality of superconducting coils and generates a dipole magnetic field; and
a superconducting quadrupole coil that is formed of the plurality of superconducting coils and generates a quadrupole magnetic field,
wherein the superconducting dipole coil and the superconducting quadrupole coil are arranged coaxially with each other.

10. A superconducting accelerator comprising a plurality of superconducting coil apparatuses, each of which is the superconducting coil apparatus according to claim 1,
wherein a beam trajectory for accelerating a particle beam is formed by the plurality of superconducting coil apparatuses.

11. A particle beam therapy apparatus comprising the superconducting accelerator according to claim 10,
wherein the superconducting accelerator is configured to accelerate the particle beam in such a manner that a lesion site is irradiated with the particle beam for treatment.
